(19) United States
(12) Patent Application Publication
(10) Pub. No.: US 2002/0127589 A1
(43) Pub. Date: Sep. 12, 2002

(54) Microarray and microarray substrate

(76) Inventors: Keiichi Sato, Kanagawa (JP); Toshiki Morita, Kanagawa (JP)

Correspondence Address: Pillsbury Winthrop LLP, Suite 200, 11975 El Camino Real, San Diego, CA, 92130, US

(21) Appl. No.: 10/077,649

(22) Filed: Feb. 14, 2002

(30) Foreign Application Priority Data
Mar. 8, 2001 (JP) 64918/2001

(51) Int. Cl. C12Q 1/68; C12M 1/34; G01N 33/543

(52) U.S. Cl. 435/6; 435/287.2; 436/518

(57) ABSTRACT

To provide microarray capable of readily and securely making the shape of spot of probe DNA to be fixed into a desired shape.

A microarray 2 of which substrate is a slide glass has a hydrophilic region 3 where the surface is hydrophilic and a probe DNA is fixed, and a hydrophobic region 4 where a probe DNA is not fixed and the surface is hydrophobic, around the hydrophilic region 3. When a solution containing the probe DNA is dropped by a spotter 5, the solution spreads in the hydrophilic region 3 while being prevented from further spreading by the hydrophobic region 4. As a result of this, it is possible to arbitrarily determine the shape of the spot, which is the hydrophilic region 3.

### PRIORITY INFORMATION

This application claims priority to Japanese Application Serial No. 2001-64918, filed Mar. 8, 2001.

### BACKGROUND OF THE INVENTION

The present invention relates to a microarray and a microarray substrate for analyzing whether or not a target sequence exists in a sample biopolymer by using a hybridization reaction between the sample biopolymer and a probe biopolymer.

Conventionally, for the purpose of characterizing or fractionating molecules in a living body, particularly for the purpose of detecting a target DNA or detecting presence/absence of a gene DNA, methods in which a nucleic acid or a protein having a known sequence serving as a probe is caused to hybridize with a sample DNA labeled with a fluorescent agent (generally, sample biopolymer) have been widely used. To be more specific, these methods are conducted by using a DNA chip (generally, microarray) in which a probe DNA (generally, probe biopolymer) is fixed on a slide glass. First, drop a solution containing a sample DNA labeled with a fluorescent agent on a slide glass on which a probe DNA is fixed, then put a cover glass thereon to allow them to hybridize with each other. Since the sample DNA is fixed together with the probe DNA as the sample DNA bounds to the probe DNA, it is possible to detect the hybridized sample DNA by exciting the fluorescent agent with which the fixed sample DNA is labeled, with exciting light from a light source after cleaning the slide glass and detecting the fluoresce of the emitted light.

The DNA chip is created in the manner as follows:

(1) Apply an agent for making a probe biopolymer fixed such as poly-L-lysine on the surface of a slide glass.

(2) Drop a solution containing a probe DNA in the form of spots in a predetermined layout using a spotter having a finely machined pin to fix the probe DNA.

Besides this manner, an inkjet system or a nozzle system, and a system that uses a surface-treated slide glass are also applicable.

Merely dropping a solution containing a probe DNA as is in the prior art produces a spot shape of distorted circle. For this reason, it is necessary to leave a space between adjacent spots with a certain allowance so that the respective probe DNAs do not mix with each other. Furthermore, since there is no guarantee that the shape of a spot is circular, the detection is carried out only in a narrow part in the center of the spot.

Furthermore, in the case where a surface-treated slide glass is used, it takes much time and effort for operation of machining the glass.

### SUMMARY OF THE INVENTION

In consideration of the above problems, it is an object of the present invention to provide a microarray and a microarray substrate capable of shaping a spot of probe DNA to be fixed, into the desired shape easily and reliably.

A microarray according to the present invention has on its surface a hydrophilic region where a probe biopolymer is fixed, and a hydrophobic region where a probe biopolymer is not fixed, around the hydrophilic region.

Further, when the hydrophilic region is circular, a stable spot shape can be obtained.

Further, when the hydrophilic region is nearly rectangular, an effective available area for detection on the microarray increases, and in the case where the shape is rectangular, it provides an advantage in carrying out analyzing operation after reaction over the case where the shape is circular.

Further, since the agent for making a probe biopolymer fixed is formed on the surface of the hydrophilic region while the agent for making a probe biopolymer fixed is not formed on the surface of the hydrophobic region around the hydrophilic region, it is possible to make the region where the probe biopolymer is fixed into a desired shape more securely.

Furthermore, a microarray substrate according to the present invention has on its surface a hydrophilic region where a probe biopolymer is fixed, and a hydrophobic region where a probe biopolymer is not fixed, around the hydrophilic region.

Furthermore, a microarray substrate according to the present invention has on its surface a hydrophilic region where an agent for making a probe biopolymer fixed is formed, and a hydrophobic region where the agent for making a probe biopolymer fixed is not formed, around the hydrophilic region.

### BRIEF DESCRIPTION OF THE DRAWINGS

FIG. 1 is a view showing a configuration of a microarray according to an embodiment of the present invention; and

FIG. 2 is a view for explaining a method of producing a microarray according to an embodiment of the present invention.

### DESCRIPTION OF THE PREFERRED EMBODIMENTS

In the following, a preferred embodiment of the present invention will be explained in detail with reference to the attached drawings.

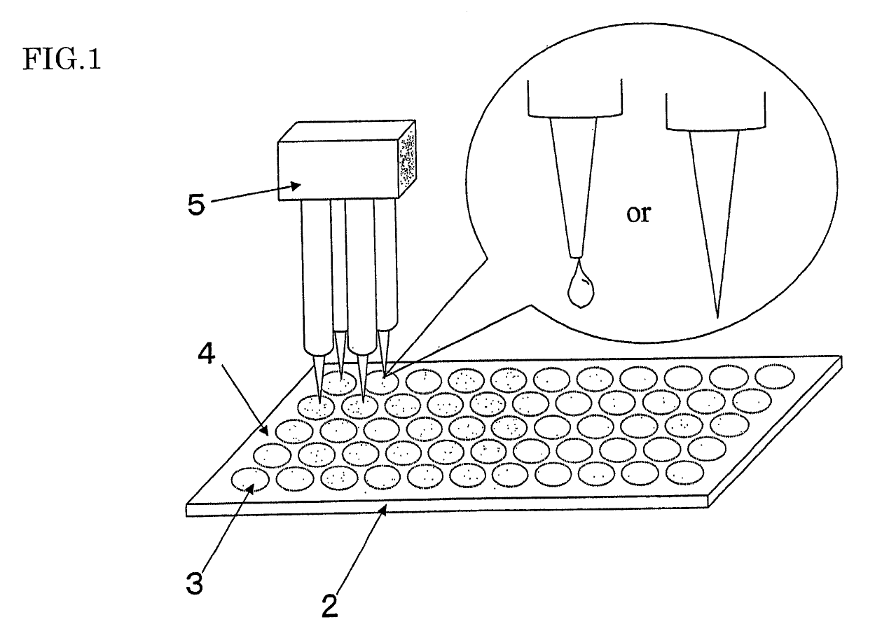

FIG. 1 is a view showing a configuration of a microarray according to an embodiment of the present invention. A microarray 2 of which substrate is a slide glass has a hydrophilic region 3 where the surface is hydrophilic and a probe DNA is fixed, and a hydrophobic region 4 where a probe DNA is not fixed and the surface is hydrophobic, around the hydrophilic region 3. In this way, the hydrophilic region 3 and the hydrophobic region 4 are selectively provided on the surface of the microarray 2, and the probe DNA is fixed in the hydrophilic region 3. Therefore, when a solution containing the probe DNA is dropped by a spotter 5, the solution spreads in the hydrophilic region 3 while being prevented from further spreading by the hydrophobic region 4. As a result of this, it is possible to arbitrarily determine the shape of the spot, which is the hydrophilic region 3. For example, the shape of the spot may be nearly round or may be substantially rectangular when it is made into a usual circular shape. Almost no restricting condition is provided for the shape. In the case where the shape of the spot is rectangular, the region to be detected increases so that it is possible to reduce an idle area, which is not used for detection. Even in the case where the shape of the spot is circular, the size can be accurately set into an arbitrary and predetermined size, so that it is possible to reduce an interval between spots in comparison with the prior art. Furthermore, since it is known that the shape is nearly complete round, the region to be detected can be a large region spreading to the vicinity of the edge of circumference. This, in turn, means that it is possible to provide a number of spots by making the shape of the spot small and thereby reducing the intervals. Furthermore, also as for the configuration of the spotter 5, since it is not necessary to employ a special structure for the purpose of making the shape in which the dropped solution spreads into a special shape, it is possible to simplify the structure by only controlling the amount of solution to be dropped.

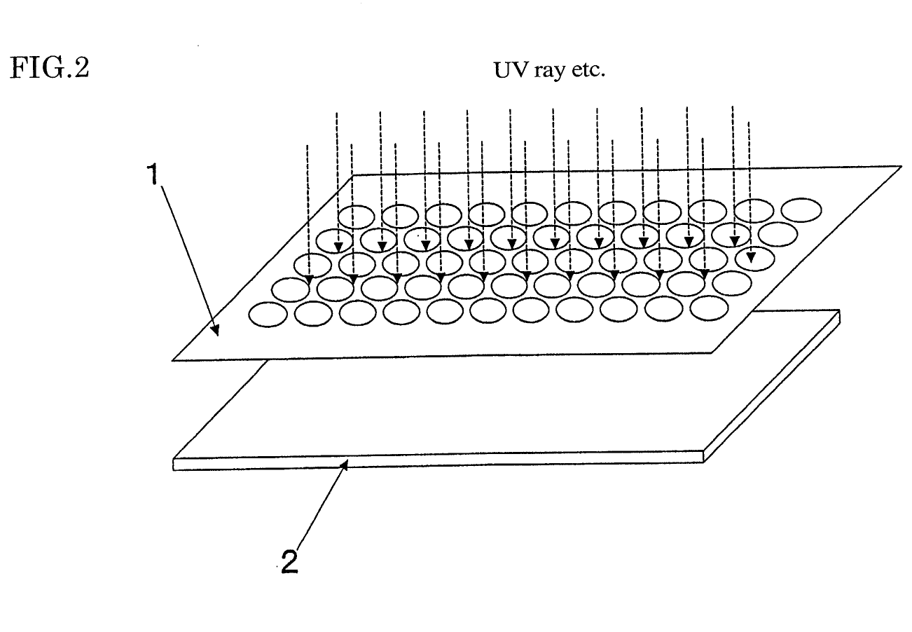

FIG. 2 is a view for explaining a method of producing a microarray according to an embodiment of the present invention. In this context, the explanation is made for the case where hydrophilic property is selectively imparted to a predetermined region of an ordinary slide glass by photocatalytic technique to render the microarray 2.

(1) Form a thin film including a photocatalytic semiconductor material on the entire surface of a slide glass, which is a substrate to become the microarray 2. The photocatalytic semiconductor material is selected from the group consisting of TiO₂, ZnO, SnO₂, SrTiO₃, WO₃, Bi₂O₃ and Fe₂O₃. (For further information, see Japanese Patent Publication No.2756474.)

(2) Change the property of the thin film of photocatalytic semiconductor material formed in the hydrophilic region 3 into hydrophilic by irradiating the microarray 2 with an ultraviolet ray via a mask 1 which selectively allows the ultraviolet ray to pass through in the region corresponding to the hydrophilic region 3 (a mask formed so that a hole is pierced in the position corresponding to the hydrophilic region 3) to thereby irradiate the hydrophilic region 3 with the ultraviolet ray in the formed thin film of photocatalytic semiconductor material.

(3) Fix a probe DNA in the hydrophilic region 3.

It is noted that the present invention is not limited to the above embodiment.

Hydrophilic property may be imparted by applying a hydrophilic paint instead of using the photocatalytic semiconductor material.

The base material for a microarray substrate is not limited to those made of glass. Any materials can be used insofar as the hydrophilic region and the hydrophobic region can be formed on their surface. For example, also plastic, metal and the like are applicable, with those not having biochemical activities being more preferred.

In the drawing as described above, all of the regions other than the hydrophilic regions of the microarray is made into hydrophobic regions, however, it is not necessary to make all of the remaining regions into the hydrophobic regions as far as the vicinity of hydrophilic regions are made into hydrophobic regions.

In fixing the probe DNA, an agent for making a probe DNA fixed may be formed on the surface of the substrate prior to dropping the solution containing the probe DNA to fix the probe DNA, or a solution containing both the agent for making a probe DNA fixed and the probe DNA may be dropped to fix the probe DNA. In either case, the probe DNA is selectively fixed to the hydrophilic region 3.

As described above, according to the present invention, it is possible to make the shape of the spot of probe DNA fixed to the microarray substrate into a desired shape readily and securely. Therefore, it is possible to increase the number of spots of probe DNA fixed on the microarray, as well as making the structure of the spotter simple.

What is claimed is:

1. A microarray having on its surface a hydrophilic region where a probe biopolymer is fixed, and a hydrophobic region where a probe biopolymer is not fixed, around the hydrophilic region.

2. The microarray according to claim 1, wherein the hydrophilic region is circular.

3. The microarray according to claim 1, wherein the hydrophilic region is nearly rectangular.

4. The microarray according to claim 1, wherein an agent for making a probe biopolymer fixed is formed on the surface of the hydrophilic region, and the agent for making a probe biopolymer fixed is not formed on the surface of the hydrophobic region, around the hydrophilic region.

5. A microarray substrate having on its surface a hydrophilic region where a probe biopolymer is fixed, and a hydrophobic region where a probe biopolymer is not fixed, around the hydrophilic region.

6. A microarray substrate having on its surface a hydrophilic region where an agent for making a probe biopolymer fixed is formed, and a hydrophobic region where the agent for making a probe biopolymer fixed is not formed, around the hydrophilic region.
